(19) United States
(12) Patent Application Publication
(10) Pub. No.: US 2015/0282779 A1
(43) Pub. Date: Oct. 8, 2015

(54) Treating an Ischemic Stroke

(71) Applicants: Yu Deuerling-Zheng, Forchheim (DE); Klaus Klingenbeck, Aufsess (DE)

(72) Inventors: Yu Deuerling-Zheng, Forchheim (DE); Klaus Klingenbeck, Aufsess (DE)

(21) Appl. No.: 14/243,374

(22) Filed: Apr. 2, 2014

(51) Int. Cl. A61B 6/00; A61M 5/00; A61B 6/03

(52) U.S. Cl. CPC A61B 6/504; A61B 6/032; A61M 5/007; A61B 6/501; A61B 6/5217; A61B 6/4441; A61B 6/5205

(57) ABSTRACT

Systems and methods for treating an ischemic stroke are provided. In one aspect, a non-contrast scan is performed on a patient with a C-arm computed tomography (CT) imaging device in an interventional suite. A contrast agent is injected into the patient. A plurality of contrast scans is performed on the patient with the C-arm CT imaging device. Three-dimensional vascular images are reconstructed using the non-contrast scan and the plurality of contrast scans. A location of an ischemic stroke is determined from the reconstructed three-dimensional data sets and calculation of cerebral blood flow, mass transit time, time to peak, or a combination thereof. The ischemic stroke is treated in the interventional suite.

### TECHNICAL FIELD

The present embodiments relate to treating a stroke, and, in particular, analyzing and treating an ischemic stroke in an interventional environment or suite.

### BACKGROUND

Cerebrovascular disorders represent a group of diverse vascular diseases that include atherosclerosis, ischemic stroke, aneurysms, and arteriovenous malformations. Due to the high prevalence and significant impact of these diseases, multiple interventional treatment options have been developed over the recent years. Functional, (i.e., physiological imaging), plays an important role in the diagnosis and treatment decisions in many neurovascular diseases, such as ischemic stroke or vasospasm following hemorrhage of an aneurysm. While perfusion imaging is a well-established diagnostic imaging technique, it has not been available in an interventional suite.

Instead, to get information on functional changes during the interventions today, the patient has to be transported from the interventional suite to an external appropriate diagnostic faculty. Diffusion and perfusion weighted MRI techniques have revolutionized the role of magnetic resonance imaging (MRI) in the evaluation of patients with acute cerebrovascular disease. These techniques allow determination of cerebral blood flow (CBF), mean transit time (MTT), and cerebral blood volume (CBV) so that it is possible to differentiate brain tissue that is still viable from that which has been irreversibly injured because of inadequate blood flow. Such MR imaging is, however, time-consuming, not widely available and most importantly cannot be done in an environment that is optimal for therapeutic interventions.

Currently available computed tomography (CT) scanners also allow rapid and accurate determination of these functional parameters, e.g., CBF, MTT, and CBV. While CT techniques are widely available, these techniques do not exist in an environment that is optimal for the type of therapeutic interventions required for the treatment of most cerebrovascular conditions. These same limitations also apply to other techniques, such as Positron Emission Tomography (PET) and Single Photon Emission Computed Tomography (SPECT), which can be used to measure the physiological aspects of the brain circulation.

Therefore, to have functional information about tissue health available within an interventional suite would help to optimize management of patients with neurovascular diseases.

### SUMMARY

Systems and methods are provided for analyzing and treating an ischemic stroke in an interventional suite. In these embodiments, a patient may be examined and treated within a single location, minimizing movement of the patient and reducing overall time to treatment.

In one aspect, a non-contrast scan is performed on a patient with a C-arm computed tomography (CT) imaging device in an interventional suite. A contrast agent is injected into the patient. A plurality of contrast scans is performed on the patient with the C-arm CT imaging device. Three-dimensional vascular images are reconstructed using the non-contrast scan and the plurality of contrast scans. A location of an ischemic stroke is determined from the reconstructed three-dimensional data sets and calculation of cerebral blood flow, mass transit time, time to peak, or a combination thereof. The ischemic stroke is treated in the interventional suite.

In another aspect, a preliminary non-contrast scan is performed on a patient with a C-arm CT imaging device in an interventional suite. The absence of an intercerebral hemorrhage is confirmed from the preliminary non-contrast scan. An additional non-contrast scan is performed on the patient with the C-arm CT imaging device in the interventional suite. A contrast agent is injected into the patient. A plurality of contrast scans are performed on the patient with the C-arm CT imaging device, wherein each contrast scan in the plurality of contrast scans is completed in less than 3 or 5 seconds. Three-dimensional vascular images are reconstructed from the non-contrast scan and the plurality of contrast scans. A location of an ischemic stroke is determined from the reconstructed three-dimensional data sets and the calculation of cerebral blood flow, mass transit time, time to peak, or a combination thereof. The ischemic stroke is treated in the interventional suite. A follow-up contrast scan is performed on the patient with the C-arm CT imaging device in the interventional suite, wherein a time between the non-contrast scan and the performing the follow-up contrast scan is less than one hour.

In yet another aspect, the interventional environment system includes a C-arm computed tomography imaging device, an interventional suite housing the C-arm CT imaging device and endovascular equipment or medication for treating an ischemic stroke, and a processor, where the C-arm CT imaging device and the processor are configured to: (1) perform a non-contrast scan on a patient within the interventional suite; (2) perform a plurality of contrast scans on the patient with the C-arm CT imaging device after a contrast agent has been injected into the patient within the interventional suite; (3) reconstruct three-dimensional vascular data sets using the non-contrast scan and the plurality of contrast scans; and (4) calculate the cerebral blood flow (CBF), mass transit time (MTT), time to peak (TTP), or a combination thereof from the reconstructed three-dimensional vascular data sets, wherein the calculated CBF, MTT, TTP, or combination thereof is used to determine a location of an ischemic stroke.

The present invention is defined by the following claims, and nothing in this section should be taken as a limitation on those claims. Further aspects and advantages of the embodiments are discussed below and may be later claimed independently or in combination.

### BRIEF DESCRIPTION OF THE DRAWINGS

FIG. 1 illustrates an example flowchart for analyzing and treating an ischemic stroke in an interventional suite.

FIG. 2 illustrates an example of a C-arm CT imaging device.

### DETAILED DESCRIPTION

The embodiments described herein include methods or systems for diagnosing patients presenting with one or more symptoms which may be indicative of a stroke. However, the diseases, syndromes, conditions, and the like, and the types of examination, diagnosis, and treatment protocols described herein are by way of example, and are not meant to suggest that embodiments are limited to those named, or the equivalents thereof. As the medical arts are continually advancing, the use of the embodiments described herein may be expected to encompass a broader scope in the diagnosis and treatment of patients. For example, while the techniques below are with particular reference to the brain, it should be understood that the approach can be used with respect to other organs and body parts, and the reduction of artifacts arising from high contrast differences may likewise be mitigated.

In certain embodiments, a method of diagnosing and treating a patient for a stroke may include one or more of the following: (1) conducting a physical exam on a patient, where symptoms of a stroke are diagnosed, (2) transferring the patient to an interventional environment or suite, such as a angiography laboratory suite, (3) performing a native CT scan on the patient using a C-arm CT imaging device, and storing the image data or projections into a memory device, (4) determining if the patient is suffering from internal bleeding/hemorrhagic stroke or an ischemic stroke, (5) in a case of an ischemic stroke, performing additional scans on the patient using the C-arm CT imaging device, with or without contrast, wherein the additional image data or projections are stored on the memory device (6) analyzing the results of the plurality of CT scans to determine a location of treatment, (7) treating the patient in the interventional suite, and (8) evaluating the results of the treatment in the interventional suite using the C-arm CT imaging device. It is noted that following transfer of the patient to the interventional suite, under certain embodiments, it is possible to treat a patient's ischemic stroke within the same interventional suite as the C-arm CT device. Such a process for an ischemic stroke may limit movement of patient and reduce the amount of time between diagnosis and treatment over treatment methods using conventional CT or MRI devices.

### Physical Examination and Transfer to Interventional Suite

In certain embodiments, the method of diagnosing and treating a patient for a stroke begins with conducting a physical exam on a patient. The physical examination may be conducted following admission of the patient to an emergency room of a hospital. Alternatively, the examination may be conducted prior to the patient arriving at a hospital.

Following a diagnosis of a potential stroke, the patient is transferred to an interventional suite (e.g., an angiography laboratory suite or room) for further examination. The interventional suite may be a room that houses or contains a C-arm CT imaging device and any surgical equipment and/or medication needed for treating an ischemic stroke. Medication may include a tissue plasminogen activator (tPA) provided intravenously to dissolve a clot and improve blood flow. Surgical equipment may include endovascular equipment such as a catheter used to treat the site of a blocked blood vessel.

### Non-Contrast CT Scan and Analysis

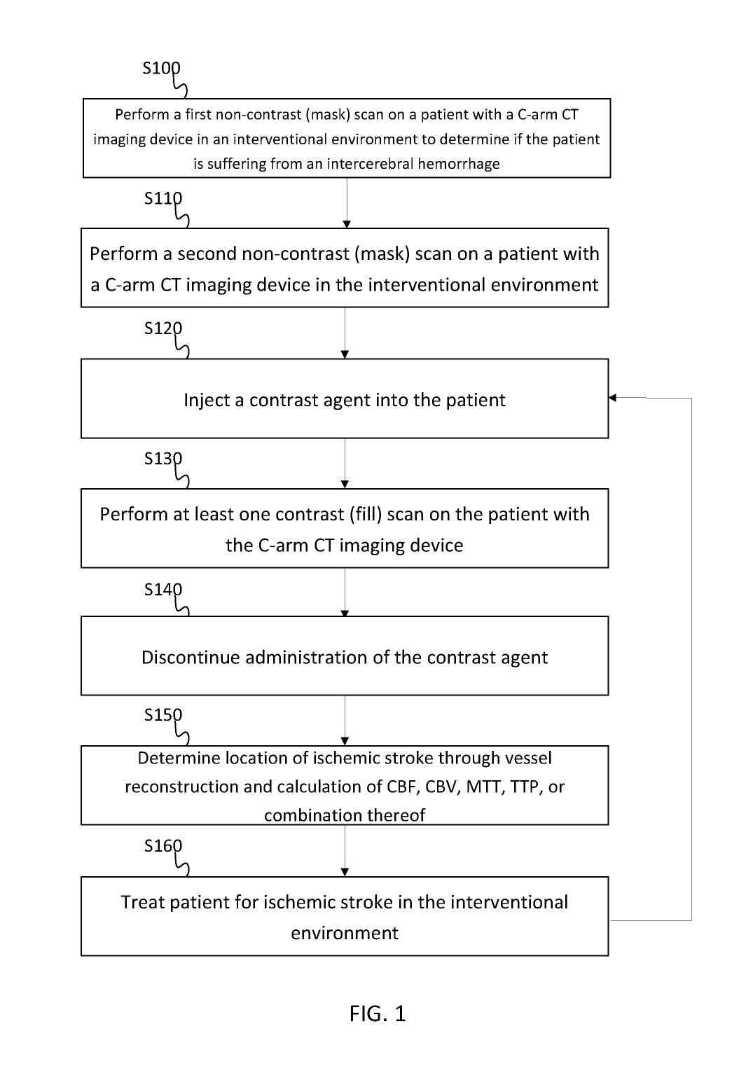

In an example shown in FIG. 1, following an initial diagnosis and movement of the patient to an interventional suite, the method may include the one or more of the following acts. In act S100, within the interventional suite, a native or non-contrast data scan may be performed on the patient using a C-arm CT imaging device. The non-contrast scan is a non-enhanced CT (“NECT”) scan conducted without the addition or injection of a contrast agent. The scan refers to the C-arm sweeping along an orbital ring about an axis of rotation around the patient to record a data set having plurality of two-dimensional slice images of the brain (i.e., a non-contrast scan data set). The 2D image recordings in the non-contrast scan data set may be analyzed to determine if the patient is suffering from an intercerebral hemorrhage (e.g., a hemorrhagic stroke). In certain embodiments, the plurality of 2D images in the non-contrast data set may be reconstructed to form three-dimensional CT data sets where the reconstruction involves processing the obtained 2D data from different angular directions. The result of the 3D reconstruction is a volumetric data set representing the x-ray attenuation values associated with a plurality of individual small volumes (voxels) of the volume that has been imaged. Techniques for reconstruction are well known, such as those described in U.S. Pat. No. 8,285,014, herein incorporated by reference in its entirety. From the reconstructed 3D data set, a 2D image of the brain may be rendered at a specified viewing angle.

An intercerebral hemorrhage may be recognized from a 2D slice image recording from the data set or a rendered 2D image because blood appears brighter than other tissue and is separated from the inner table of the skull by brain tissue. The tissue surrounding a bleed is often less dense than the rest of the brain because of edema, and therefore shows up darker on the CT scan.

In certain embodiments, in the case of internal bleeding or a hemorrhagic stroke, the patient is transferred out of the interventional suite for further treatment (e.g., medication and/or surgery). In some embodiments, following treatment to stop the bleeding, the patient may be returned to the interventional suite for further analysis. The interventional suite is provided for diagnosis and some treatments.

In an instance where a hemorrhage has been excluded in the analysis of the non-contrast CT scan, additional CT imaging may be performed with the C-arm CT imaging device to assist in the analysis and treatment of an ischemic stroke.

### Ischemic Stroke Analysis Using C-Arm CT

In certain embodiments, analysis of the ischemic stroke may be conducted with the C-arm CT imaging device by obtaining and comparing non-contrast CT data scans with contrast or enhanced CT data scans.

As discussed above, at least one initial non-contrast or NECT scan has already been obtained for the patient. In certain embodiments, this initial non-contrast scan may be used as the basis for comparison with the post-contrast injection or enhanced scan(s). In alternative embodiments, additional non-contrast or NECT scans may be obtained prior to injecting contrast. For example, as shown in FIG. 1 in act S110, in the case of an ischemic stroke, an additional non-contrast or mask scan may be performed with a C-arm CT imaging device in the interventional suite, where additional sequences of C-arm X-ray rotational images are obtained.

In act S120, following the at least one non-contrast scan with the C-arm CT device, a contrast agent may be administered to or injected into the patient either venously or arterially. In certain embodiments, the duration of the contrast injection is long enough that the parenchyma (e.g., neuron tissue of the brain) becomes saturated. Following injection of the contrast, enhanced CT images of the patient may be collected using the C-arm CT imaging device.

### Static Analysis

In an example shown in FIG. 1, in act S130, at least one post-contrast injection scan (i.e., contrast scan) is performed with the C-arm CT imaging device, where at least one additional sequence of rotational two-dimensional slice images is obtained.

In certain embodiments, a post-contrast injection scan analysis is conducted after the contrast density has stabilized and is at a steady-state level within the parenchyma. In other words, a contrast agent may be administered by a programmed injection such that, during the period of time where the contrast scan data is obtained, the density or amount of contrast agent within the parenchyma is at a steady-state level.

The contrast scan refers to the C-arm sweeping along an orbital ring about an axis of rotation around the patient to record a data set having plurality of two-dimensional slice images of the brain (i.e., a contrast scan data set). The post-contrast injection scan using the C-arm CT imaging device may be performed in the same scan direction and with the same settings (e.g., x-ray intensity, aperture, and duration) as the non-contrast scan. Additionally, the contrast scan data set may be obtained either before a catheter is introduced into the patient for potential treatment (discussed below), or after catheter introduction.

In certain embodiments, the plurality of 2D images in the contrast data set may be reconstructed to form a three-dimensional data set, where the reconstruction involves processing the obtained 2D data from different angular directions. From the reconstructed 3D data set, a 2D image of the brain may be rendered at a specified viewing angle.

After the contrast scan data set is obtained, the plurality of 2D images in the contrast data set may be reconstructed to form a three-dimensional data set, where the reconstruction involves processing the obtained 2D data from different angular directions. As discussed above, the 2D data sets may be reconstructed into three-dimensional CT data sets by well-known techniques.

It may be necessary re-register or align the relative coordinate systems of the data sets to account for any patient movement that has occurred between the scans to acquire the different sets. Such movement of the patient may occur between the recording of the non-contrast or mask and the contrast or fill data sets where the non-contrast data set is first used to exclude a cerebral hemorrhage, and a port, for example for arterial access, may then be put in place for further diagnosis and treatment. Image re-registration techniques may adjust the relative coordinate systems so as to minimize the differences between salient points of the data sets, but any technique that achieves the same effective result may be used. Rigid or affine registration may be used.

Based on the reconstructed mask/non-contrast and fill/contrast 3D data sets, cerebral blood volume (CBV) may be determined. The CBV describes the volume of blood actually present in a volume of imaged tissue. Although CBV is a functional parameter that changes due to physiological regulation processes, it may be assumed to be constant during the time required to obtain an individual acquisition. This makes it possible to calculate the CBV from only two measurements: a base-line, non-contrast/mask scan acquired before contrast administration and a contrast/fill scan after a contrast injection. That is, the reconstructed non-contrast data set and the contrast data set may be subtracted from each other so as to produce a data set of the contrast enhancement of the plurality of voxels due to the contrast agent. The difference represents the blood in the scanned region, where the volume of the blood is calculated from the known voxel size.

This process may be termed digital subtraction angiography (DSA), and here the process is performed volumetrically with the CT-like volume sets. Segmentation of the contrast data set may also be performed so as to permit the larger blood vessels in the data set to be segmented out (excised) from the data set. Segmentation may also be done, for example, by establishing a threshold value in HU and excluding the data exceeding the threshold value. The voxel data obtained by subtraction of the non-contrast voxel data from the contrast voxel data may be termed a functional CT image as the contrast difference (enhancement) is attributed to the contrast agent and representative of the cerebral perfusion or blood flow. This permits the computation of CBV.

In certain embodiments, the location of an ischemic stroke may be identified based on the CBV calculation through an identification of regions having abnormal blood volume in comparison to a healthy brain.

### Dynamic Analysis

In certain embodiments, perfusion imaging is gathered using the C-arm CT imaging system and a perfusion analysis is performed. Through the use of improved data processors and faster C-arm scan times, it may be possible to use the C-arm CT system to perform a perfusion scan to determine dynamic properties such as the flow of blood (or lack thereof) through the brain.

For example, as shown in FIG. 1 in act S140, following the completion of the C-arm CT contrast scans, administration of the contrast agent may be discontinued. In act S150, the voxel data from the non-contrast/mask scan(s) and the plurality of contrast scans may be used to reconstruct a three-dimensional vascular image, forming 3D volumetric dynamic angiography or vessel reconstruction. The reconstructed 3D data sets may be analyzed to determine cerebral blood flow (CBF), cerebral blood volume (CBV), mass transit time (MTT), time to peak (TTP), or a combination thereof.

As discussed above, CBV describes the volume of blood actually present in a volume of imaged tissue. Mean transit time (MTT) measures the time required for blood to pass through a defined amount of tissue. Based on known injection scan times and contrast injection rates or tracers injected in the contrast, imaged tissue within reconstructed 3D data sets may be analyzed to determine the MTT for a specific tissue segment. Additionally, the MTT for the specific tissue segment may be compared with known transit times for healthy tissue segments. Abnormal MTT (e.g., slower than normal transit times) may indicate a location of the ischemic stroke.

Time-to-peak (TTP) measures the time interval between administration of a contrast agent and the time it reaches its highest concentration in a specific area of interest. Similar to MTT, based on known injection scan times and contrast injection rates, tissue within reconstructed 3D data sets may be analyzed to determine the TTP for a specific tissue segment. Ischemic locations may be identified by a delay of a contrast tracer arrival and an increase in the TTP.

Cerebral blood flow (CBF) measures the blood supply to a segment of the brain in a given time. It may be calculated from the cerebral blood volume divided by the mean transmit time for a defined segment of tissue. Based upon the calculated CBF, the location of an ischemic stroke may be determined. For example, in an adult, a healthy CBF is approximately 50 to 54 milliliters of blood per 100 grams of brain tissue per minute. Too little blood flow (ischemia) is generally identified as blood flow rates below 18 to 20 ml per 100 g per minute.

In certain embodiments, the dynamic perfusion analysis includes at least one non-contrast or mask C-arm CT scan and at least one post-contrast injection or fill C-arm CT scan. In certain embodiments, a plurality of contrast scans (i.e., more than one post-contrast injection or fill scan) is performed using the C-arm CT imaging system. In some examples, the plurality of contrast scans performed includes at least 2, 3, 4, 5, 6, 7, 8, 9, or 10 fill scans to gather post-contrast injection imaging data.

In certain embodiments, the direction and/or settings of the contrast scan(s) is in the same first or forward direction as the non-contrast scan(s). Alternatively, the direction of at least one contrast scan is in a second, backwards or opposite reverse direction of the non-contrast scan. In other words, the C-arm CT device may operate bi-directionally. For example, after contrast injection, consecutive forward and backward scan runs of the C-arm CT device may be acquired to measure the dynamic enhancement and to sample the contrast enhancement at different points in time.

Unlike the static analysis, the timing of the contrast scans for perfusion data may occur prior to achieving saturation where the density of the contrast agent is substantially constant. For example, in a dynamic analysis, at least one contrast scan may be obtained immediately following injection of the contrast agent (e.g., <10 seconds after injection). Contrast scans may also be performed in the time period leading up to the steady state density of the contrast agent. Additionally, contrast scans may be performed following steady state of the contrast agent. Contrast scans may be performed after injection stops and as contrast agents leave the scanned region (i.e., post steady state).

In certain embodiments, the speed of the non-contrast and contrast scans with the C-arm CT device is helpful for collecting dynamic perfusion data. Additionally, and in conjunction with a higher-speed C-arm CT device, an improved processor may be necessary to collect the image data or projections being generated at a quicker rate.

For example, one complete scan of the C-arm CT device (e.g., a 200 degree gantry rotation) may be conducted in less than 5 seconds. In other words, a set of image data or projections for the complete scan may be collected in less than 5 seconds. In certain embodiments, a higher-speed C-arm CT device may complete one non-contrast or contrast scan in less than 3 seconds, less than 2 seconds, less than 1 second, less than 0.5 seconds, or less than 0.1 seconds. In other embodiments, the C-arm CT device may complete one non-contrast or contrast scan in approximately 5 seconds, approximately 3 seconds, approximately 2 seconds, approximately 1 second, approximately 0.5 seconds, approximately 0.1 seconds, between 0.1-5 seconds, between 1-3 seconds, between 2-3 seconds, between 1-2 seconds, between 0.5-1 second, or between 0.1-1 second. A single complete scan provides sufficient data to perform computed tomography for forming a set of data representing the volume of the scan region.

In certain embodiments, each additional scan may be conducted within a short period of time following completion of the previous scan. In other words, there may be a short pause between rotations of the C-arm CT device. In certain embodiments, the short pause between rotations is between 1-5 seconds. In other embodiments, the pause between rotations is approximately 2 seconds, approximately 1 second, approximately 0.5 seconds, or approximately 0.1 seconds.

During each rotation, the C-arm CT device may acquire between 50-500 projections. In certain embodiments, the C-arm CT device may acquire between 100-200 projections, or between 100-150 projections. In other embodiments, during each rotation, the C-arm CT device may acquire between 50-100 projections per second, or between 50-75 projections per second. In certain embodiments, the projections may be acquired at 70 kilovoltage peak (kVp) and 1.2 Gray (Gy)/frame dose level, with automatic exposure control enabled for the duration of the acquisition with a bit-depth of 14 bits.

At each point in time, the scans may be used to reconstruct the corresponding CT angiography (CTAs) that represents the filling of the vasculature at that particular point in time, forming 3D volumetric dynamic angiography. The reconstruction process may involve the use of a standard filtered back-projection algorithm, resulting in at least one base-line volume and a plurality of contrast-enhanced volumes, each of which individually represents a composite of the contrast dynamics occurring within each contrast scan. An in-plane Gaussian kernel (e.g., SD=1 mm) and an axial moving average filter (e.g., 5 mm) may be used to improve the signal-to-noise ratio of the parenchyma. Additionally, to correct for any motion, each volume may be registered with the appropriate baseline volume, that is, forward or backward. After motion correction, each voxel may be interpolated to a temporal resolution of 1 volume per second by use of a cubic spline interpolation method and then archived in a standard DICOM CT perfusion format. Further, through the use of contrast enhancement curves for each voxel and known algorithms from conventional CT analyses, the dynamic perfusion values for CBF, MTT, TTP, and CBV may be determined.

In certain embodiments, the elapsed time between the non-contrast scan and the determining of the location of the ischemic stroke is less than six hours, less than four hours, less than two hours, or less than one hour.

### Treatment and Follow-up

In an example shown in Figure, in act S160, based on the analysis, the patient may be treated for an ischemic stroke within the same interventional or angiography suite as the C-arm CT analysis.

Following the analysis of the non-contrast and/or contrast scans; a determination may be made upon the location and severity of the ischemic stroke. As mentioned above, the determination of the location may be based upon calculations CBF, CBV, MTT, TTP, or a combination thereof. In some embodiments, the location of the ischemic stroke is determined based upon the determined CBF and CBV. Since the patient is already located in the interventional suite, treatment (e.g., stenting or thrombolysis) of the patient may commence quickly following the analysis. Additionally, the patient may not even need to transfer to a different table for treatment within the interventional suite.

In certain embodiments, the elapsed time between the non-contrast scan and the treatment of the ischemic stroke is less than six hours, less than four hours, less than two hours, or less than one hour.

Following or concurrently with the treatment process, additional scans may be conducted with the C-arm CT device so as to obtain another set of data, and the remaining steps of the method are performed so as to evaluate or document the results of the treatment. For example, as shown in FIG. 1, the process may be returned to act S120, if needed, so as to perform additional contrast scans and obtain additional sets of voxel data. The additional acts S130, S140, and S150 may also be performed so as to evaluate or document the results of the treatment.

In certain embodiments, a follow-up contrast scan is conducted with the C-arm CT device in the interventional suite immediately following the conclusion of treatment (e.g., less than 10 minutes, less than 5 minutes, or less than 1 minute of treatment). In other embodiments, a follow-up contrast scan is performed concurrently with (e.g., prior to the conclusion of the treatment) in order to assess the progress of the treatment. Rather than CT-like scans, fluoroscopy scanning may be provided to monitor the treatment.

In certain embodiments, the method as described above may be performed while the patient remains in the treatment room and on the support for the C-arm X-ray apparatus, so that both diagnosis, treatment, and follow up evaluation may be performed without moving the patient between diagnostic or treatment equipment suites. This may minimize the time between the diagnosis and treatment steps, which has been shown to have a beneficial effect on clinical outcomes. Therefore, it may be possible to save millions of brain cells based upon the improved timing from diagnosis to treatment.

In certain embodiments, the elapsed time between the non-contrast scan and the follow-up contrast scan or the follow-up treatment of the ischemic stroke is less than six hours, less than four hours, less than two hours, or less than one hour.

### C-Arm CT Devices

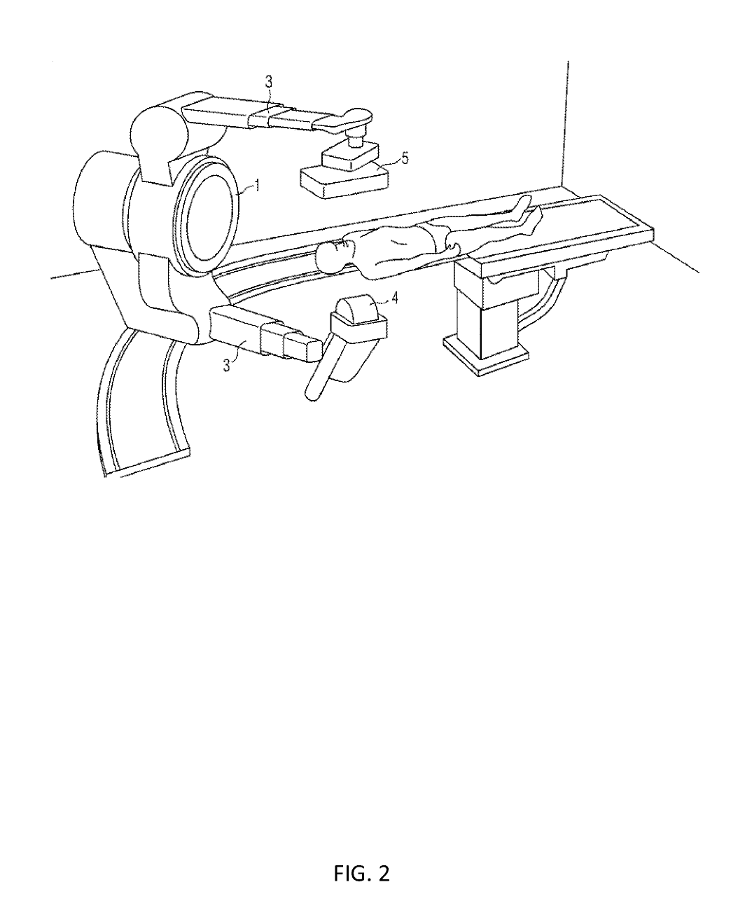

FIG. 2 depicts one embodiment of a C-arm CT imaging device useful in obtaining the non-contrast and contrast scans described in the embodiments above. As shown in FIG. 2, the C-arm CT imaging device may include a drum 1, and arms 3 that support an emitter 4 and a detector 5 (e.g., a flat-screen detector). The arms 3 may be configured to be adjustable lengthwise, so that the emitter 4 and the detector 5 may be positioned optimally in the ring structure. In certain embodiments, the C-arm supporting an x-ray source and an associated detector may be pivotably attached in a movable fashion to a displaceable unit (e.g., FIG. 2). A C-arm of an x-ray device may be moved on a buckling arm robot. The robot arm allows the x-ray source and the x-ray detector to move on a defined path around the patient. During acquisition of the non-contrast and contrast scans, the C-arm is swept around the patient. During the contrast scans, contrast agent may be injected into one of the vertebral or carotid artery.

While C-arm x-ray devices are designed primarily for the flexible but static acquisition of projection recordings, computed tomography devices operating with x-ray radiation sources traveling along an orbital ring about an axis of rotation are used to generate sectional image recordings. In certain areas, computed tomography devices may be replaced by C-arm x-ray devices with an extended functional scope. These C-arm x-ray devices may also generate sectional images, called CT-like scans. Sectional images are generated from image sequences obtained using a recording system that may be displaced along a trajectory. The reconstruction quality is not as good as may be achieved using a computed tomography device. Also, the image recording system may not be rotated completely.

High rotation speeds of the image recording system may be attained with the C-arm CT device. For example, one complete scan (e.g., a 200 degree gantry rotation) of the high speed C-arm CT device may be conducted in less than 5 seconds, less than 3 seconds, less than 2 seconds, less than 1 second, less than 0.5 seconds, less than 0.1 seconds, approximately 5 seconds, approximately 3 seconds, approximately 2 seconds, approximately 1 second, approximately 0.5 seconds, approximately 0.1 seconds, between 0.1-5 seconds, between 1-3 seconds, between 2-3 seconds, between 1-2 seconds, between 0.5-1 second, or between 0.1-1 second.

During each rotation, the high speed C-arm CT device may acquire between 50-500 projections, between 100-200 projections, or between 100-150 projections. In other embodiments, during each rotation, the C-arm CT device may acquire between 50-100 projections per second, or between 50-75 projections per second. In certain embodiments, the projections may be acquired at 70 kVp and 1.2 Gy/frame dose level, with automatic exposure control enabled for the duration of the acquisition with a bit-depth of 14 bits.

Due to the high rotation speeds, reconstruction artifacts that result due to the movement of the patient or organs (e.g., heart) during a recording are thus reduced to a minimum. The fact that the system may be constructed in a very rigid fashion provides that the rotation is highly reproducible. This allows the initially measured circuit of beam focus and detector to be repeated very accurately, so that a very precise reconstruction is achieved by the stored projection matrices.

Post-processing of the non-contrast and contrast scans may be performed using commercial software (e.g., CT perfusion 4, GE Healthcare, Pewaukee, Wis.) and/or other software for the C-arm CT studies. After reconstruction and subtraction of the non-contrast run and the contrast run, an algorithm may be applied to further segment out air and bone from the image volume. In post-processing, vascular inputs may be selected manually or automatically. In the C-arm CT post-processing, the steady-state arterial and venous input functions may be calculated from an automated histogram analysis of the vessel tree. A final scaling may be then applied to account for the arterial input, as well as other physiologic values (e.g., hematocrit), before a smoothing filter is applied to reduce pixel noise.

The acts of FIG. 1 may be performed by a processor or under the control of a processor. For example, the processor loads scan data (e.g., volume data sets) and performs the static and/or dynamic studies. The processor calculates the values of the various functional parameters, such as CBV, CBF, MTT, or TTP. The combination of hardware and software to accomplish the tasks described herein is termed a system. Where otherwise not specifically defined, acronyms are given their ordinary meaning in the art.

The instructions for implementing processes or methods of the system, may be provided on non-transitory computer-readable storage media or memories, such as a cache, buffer, RAM, FLASH, removable media, hard drive, or other computer readable storage media. Computer readable storage media include various types of volatile and non-volatile storage media. The functions, acts, or tasks illustrated in the figures or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Likewise, processing strategies may include multiprocessing, multitasking, parallel processing and the like.

The above describes embodiments of the invention and does not limit the invention. Any modification, equivalent replacement, improvement, etc. made within the spirit and principle of the invention is included in the protective scope.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method of analyzing and treating an ischemic stroke, the method comprising:
performing a non-contrast scan on a patient with a C-arm computed tomography (CT) imaging device in an interventional suite, the interventional suite housing the C-arm CT imaging device and endovascular equipment or medication for treating an ischemic stroke;
injecting a contrast agent into the patient;
performing a plurality of contrast scans on the patient with the C-arm CT imaging device, the plurality of contrast scans occurring after injection of the contrast agent;
reconstructing three-dimensional data sets from the non-contrast scan and the plurality of contrast scans;
determining a location of an ischemic stroke from the reconstructed three-dimensional data sets and calculation of cerebral blood flow (CBF), mass transit time (MTT), time to peak (TTP), or a combination thereof; and
treating the ischemic stroke in the interventional suite.

2. The method of claim 1, further comprising, prior to performing the non-contrast scan, performing a preliminary non-contrast scan on a patient with the C-arm CT imaging device in the interventional suite;
confirming an absence of an intercerebral hemorrhage from the preliminary non-contrast scan.

3. The method of claim 1, further comprising:
performing a follow-up contrast scan on the patient with the C-arm CT imaging device in the interventional suite.

4. The method of claim 3, wherein the follow-up contrast scan is conducted immediately following the treating.

5. The method of claim 3, wherein the follow-up contrast scan is conducted prior to conclusion of the treating.

6. The method of claim 3, further comprising:
conducting follow-up treatment of the ischemic stroke in the interventional suite.

7. The method of claim 6, wherein a time between the non-contrast scan and the conducting follow-up treatment of the ischemic stroke is less than two hours.

8. The method of claim 6, wherein a time between the non-contrast scan and the conducting follow-up treatment of the ischemic stroke is less than one hour.

9. The method of claim 1, wherein the determining of the location of the ischemic stroke is further based on calculation of cerebral blood volume (CBV).

10. The method of claim 9, wherein the determining of the location of the ischemic stroke is based on a combination of the cerebral blood flow and the cerebral blood volume.

11. The method of claim 1, wherein each contrast scan in the plurality of contrast scans is completed in less than 3 seconds.

12. The method of claim 1, wherein the non-contrast scan is performed in a forward direction, and the plurality of contrast scans is performed in both the forward direction and a backward direction, opposite the forward direction.

13. A method of analyzing and treating an ischemic stroke, the method comprising:
performing a preliminary non-contrast scan on a patient with a C-arm computed tomography (CT) imaging device in an interventional suite, the interventional suite housing the C-arm CT imaging device and endovascular equipment or medication for treating an ischemic stroke;
confirming an absence of an intercerebral hemorrhage from the preliminary non-contrast scan;
performing an additional non-contrast scan on the patient with the C-arm CT imaging device in the interventional suite;
injecting a contrast agent into the patient;
performing a plurality of contrast scans on the patient with the C-arm CT imaging device, the plurality of contrast scans occurring after injection of the contrast agent, wherein each contrast scan in the plurality of contrast scans is completed in less than 3 seconds;
reconstructing three-dimensional data sets from the non-contrast scan and the plurality of contrast scans;
determining a location of an ischemic stroke from the reconstructed three-dimensional data sets and calculation of cerebral blood flow (CBF), mass transit time (MTT), time to peak (TTP), or a combination thereof;
treating the location of the ischemic stroke in the interventional suite; and
performing a follow-up contrast scan on the patient with the C-arm CT imaging device in the interventional suite, wherein a time between the non-contrast scan and the performing the follow-up contrast scan is less than one hour.

14. An interventional environment system comprising:
a C-arm computed tomography (CT) imaging device;
an interventional suite housing the C-arm CT imaging device and endovascular equipment or medication for treating an ischemic stroke; and
a processor,
wherein the C-arm CT imaging device and the processor are configured to:
perform a non-contrast scan on a patient within the interventional suite;
perform a plurality of contrast scans on the patient with the C-arm CT imaging device after a contrast agent has been injected into the patient within the interventional suite;
reconstruct three-dimensional vascular data sets using the non-contrast scan and the plurality of contrast scans; and
calculate the cerebral blood flow (CBF), mass transit time (MTT), time to peak (TTP), or a combination thereof from the reconstructed three-dimensional vascular data sets,
wherein the calculated CBF, MTT, TTP, or combination thereof is used to determine a location of an ischemic stroke.

15. The system of claim 14, wherein the C-arm computed tomography imaging device and the processor are further configured to:
perform a preliminary non-contrast scan, prior to the non-contrast scan, on the patient with the C-arm CT imaging device in the interventional suite; and
confirm an absence of an intercerebral hemorrhage.

16. The system of claim 14, wherein the C-arm computed tomography imaging device and the processor are further configured to:
perform a follow-up contrast scan on the patient with the C-arm CT imaging device within the interventional suite following a treatment of the ischemic stroke.

17. The system of claim 14, wherein the C-arm computed tomography imaging device and the processor are further configured to:
calculate the cerebral blood volume (CBV), wherein the calculated CBV is used to determine the location of the ischemic stroke.

18. The system of claim 14, wherein each contrast scan in the plurality of contrast scans is completed in less than 3 seconds.

19. The system of claim 14, wherein a time between the non-contrast scan and the determining of the location of the ischemic stroke is less than one hour.

20. The system of claim 14, wherein a time between the non-contrast scan and the follow-up contrast scan is less than one hour.
